(19) United States
(12) Patent Application Publication
(10) Pub. No.: US 2007/0032410 A1
(43) Pub. Date: Feb. 8, 2007

(54) COMPOSITIONS AND METHODS FOR THE TREATMENT OF PSYCHIATRIC DISORDERS

(75) Inventors: Steven C. Quay, Seattle, WA (US); Alexis Kays Leonard, Maple Valley, WA (US); Henry R. Costantino, Woodinville, WA (US); Anthony P. Sileno, Brookhaven Hamlet, NY (US); Joshua O. Sestak, Kirkland, WA (US)

Correspondence Address: NASTECH PHARMACEUTICAL COMPANY INC, 3830 MONTE VILLA PARKWAY, BOTHELL, WA, 98021-7266, US

(73) Assignee: Atossa Healthcare, Inc.

(21) Appl. No.: 11/537,468

(22) Filed: Sep. 29, 2006

Related U.S. Application Data
(63) Continuation-in-part of application No. 10/879,814, filed on Jun. 28, 2004.
Application No. 10/879,814 is a continuation of application No. 09/678,591, filed on Oct. 3, 2000, now Pat. No. 6,894,026.
Application No. 09/678,591 is a continuation-in-part of application No. 09/481,058, filed on Jan. 11, 2000, now abandoned.

(51) Int. Cl. A61K 38/22

(52) U.S. Cl. 514/9; 514/12

(57) ABSTRACT

Methods and compositions containing oxytocin or an oxytocin analog, specifically carbetocin, are provided for the prevention and treatment of autism spectrum disorders, related disorders and symptoms of such disorders. The methods and compositions of the invention are effective in the treatment of social withdrawal, eye contact avoidance, repetitive behaviors, anxiety, attention deficit, hyperactivity, depression, loss of speech, verbal communication difficulties, aversion to touch, visual difficulties, comprehension difficulties, and sound and light sensitivity. Additional compositions and methods are provided which employ oxytocin or an oxytocin analog in combination with a secondary or adjunctive therapeutic agent to yield more effective treatment tools against autism spectrum disorders and related disorders.

### RELATED APPLICATIONS

This application is a continuation-in-part and claims the benefit under 35 U.S.C. § 120 of U.S. patent application Ser. No. 10/879,814, filed Jun. 28, 2004, which is a continuation of U.S. patent application Ser. No. 09/678,591, filed Oct. 3, 2000, which is a continuation in part of U.S. patent application Ser. No. 09/481,058, filed Jan. 11, 2000, now abandoned. These cited applications are herein incorporated by reference in their entirety.

### TECHNICAL FIELD

The present invention relates to methods and compositions for the treatment of neurological and psychiatric disorders. In specific embodiments, the invention relates to the treatment of neurological and psychiatric disorders using carbetocin and related oxytocin analogs.

### BACKGROUND

Autism spectrum disorders are a group of diseases characterized by varying degrees of impairment in communication skills, social interactions, and restricted, repetitive and stereotyped patterns of behavior. The difference in the diseases depends on the time of onset, the rate at which symptoms develop, the severity of symptoms, and the exact nature of the symptoms. These disorders range from mild to severe impairment and include such diseases as autism, Asperger's syndrome, PDD-NOS, Rett's disorder, childhood disintegrative disorder, semantic communication disorder, non-verbal learning disabilities, high functioning autism, hyperlexia and some aspects of attention deficit hyperactivity disorder. While the exact number of children with autism spectrum disorders is unclear, rates in localized areas of the United States vary from 3.4 children per one thousand to 6.7 children per one thousand. Further, recent studies estimate that 15,000 children aged three through five years, and 78,000 children and young adults aged six through twenty-one years in the United States have autism. Rates in Europe and Asia are similar, with as many as six per one thousand children having at least one autism spectrum disorder. Additionally, there are number of related disorders including anxiety disorders, obsessive-compulsive disorders, social deficit disorders, repetitive disorders and cognitive deficit disorders which exhibit symptoms similar to those displayed in autism spectrum disorders, greatly increasing the size of the affected population.

Characteristics of autism spectrum disorders include social withdrawal and averted gaze including an inability to make eye contact, repetitive behaviors and obsessions, stereotyped movements, anxiety, attention deficit, hyperactivity, depression, a reclusive personality, and the inability to understand feelings. Patients afflicted with autism spectrum disorders may have an aversion to physical affection or contact, ignore communication from others, or if socially engaged, demonstrate a marked inability to communicate or relate to others. Communication difficulties may manifest as a monotone voice, an inability to control the volume of their voice, echolalia or an inability to talk at all. Individuals with autism spectrum disorders may also suffer from visual difficulties, comprehension difficulties, sound and light sensitivity and mental retardation.

Children with autism spectrum disorders do not follow the typical patterns of child development. In some children, hints of future problems may be apparent from birth. In most cases, the problems in communication and social skills become more noticeable as the child lags further behind other children the same age. Some children initially develop normally and then begin to develop differences in the way they react to people and other unusual behaviors. Some parents report the change as being sudden, and that their children start to reject people, act strangely, and lose language and social skills they had previously acquired. In other cases, there is a plateau in development that becomes increasingly noticeable.

The underlying causes of autism spectrum and related disorders are unclear. Postmortem and MRI studies have implicated anomalies in many major brain structures including the cerebellum, cerebral cortex, limbic system, corpus callosum, basal ganglia, and brain stem. Other research is examining the role of neurotransmitters such as serotonin, dopamine, and epinephrine.

Currently, autism spectrum disorders are treated using applied behavior analysis or other behavior modification techniques; dietary modification such as a gluten or casein free diet, or large doses of vitamin B6 in combination with magnesium. Medications prescribed for autism address specific symptoms such as anxiety and depression and include agents such as fluoxetine, fluvoxamine, sertraline and clomipramine. Antipsychotic medications such as chlorpromazine, thioridazine, and haloperidol have been used to treat behavioral problems. Anticonvulsants such as arbamazepine, lamotrigine, topiramate, and valproic acid have been given to prevent seizures.

Unfortunately, current treatments for autism spectrum and related disorders are mainly symptomatic and have proven unsuccessful in allowing such children and adults to become symptom, or disorder, free. There is therefore an unmet need in the art for alternative treatments for autism spectrum disorders and related pathologies.

### SUMMARY OF THE INVENTION

It is an object of the present invention to provide methods and compositions for the treatment of neurological and psychiatric disorders.

It is an additional object of the present invention to provide methods and compositions for the treatment of autism spectrum disorders and disorders that include related symptoms such as developmental disorders, anxiety disorders, repetitive disorders, and cognitive deficit disorders.

It is another object of the present invention to provide novel formulations of oxytocin and related analogs including carbetocin for the treatment of autism spectrum disorders and related disorders.

It is a further object of the present invention to provide compositions and methods for treating and preventing symptoms of autism spectrum disorders and related disorders including, but not limited to, social withdrawal, eye contact avoidance, repetitive behaviors, anxiety, attention deficit, hyperactivity, depression, loss of speech, verbal communication difficulties, aversion to touch, visual difficulties, comprehension difficulties, and sound and light sensitivity.

The invention achieves these objects and satisfies additional objects and advantages by providing novel and surprisingly effective methods and compositions for treating and/or preventing autism spectrum disorders, related disorders and symptoms of such disorders using oxytocin and oxytocin analogs.

Useful oxytocin and oxytocin analogs within the formulations and methods of the invention include, but are not limited to, 4-threonine-1-hydroxy-deaminooxytocin, 9-deamidooxytocin, an analog of oxytocin containing a glycine residue in place of the glycinamide residue; 7-D-proline-oxytocin and its deamino analog; (2,4-diisoleucine)-oxytocin, an analog of oxytocin with natriuretic and diuretic activities; deamino oxytocin analog; a long-acting oxytocin (OT) analog, 1-deamino-1-monocarba-E12-[Tyr(OMe)]-OT(dCOMOT); carbetocin, (1-butanoic acid-2-(O-methyl-L-tyrosine)-1-carbaoxytocin, or, alternatively, deamino-1 monocarba-(2-O-methyltyrosine)-oxytocin [d(COMOT)]); [Thr4-Gly7]-oxytocin (TG-OT); oxypressin; Ile-conopressin; atosiban; deamino-6-carba-oxytoxin (dC60), d[Lys(8)(5/6C-Fluorescein)]VT, d[Thr(4), Lys(8)(5/6C-Fluorescein)]VT, [HO(1)] [Lys(8)(5/6C-Fluorescein)]VT, [HO(1)][Thr(4), Lys(8)(5/6C-Fluorescein)]VT, d[Om(8)(5/6CFluorescein)]VT, d[Thr(4), Om(8)(5/6C-Fluorescein)]VT, [HO(1)][Om(8)(5/6C-Fluorescein)]VT, [HO(1)][Thr(4), Om(8)(5/6C-Fluorescein)]VT, desmopressin, and 1-deamino-oxytocin in which the disulfide bridge between residues 1 and 6 is replaced by a thioether. Other useful forms of oxytocin or oxytocin analogs for use within the invention include other pharmaceutically acceptable active salts of said compounds, as well as active isomers, enantiomers, polymorphs, solvates, hydrates, and/or prodrugs of said compounds.

In exemplary embodiments, the compositions and methods of the invention employ oxytocin and/or an oxytocin analog to treat and/or prevent autism spectrum disorders, related disorders and symptoms of such disorders.

Mammalian subjects amenable for treatment using the compositions and methods of the invention include, but are not limited to, human and other mammalian subjects suffering from a psychiatric or neurological disorder including autism spectrum disorders such as autism, Asperger's syndrome, pervasive developmental disorder not otherwise specified, Rett's disorder, childhood disintegrative disorder, semantic pragmatic communication disorder, non-verbal learning disabilities, high functioning autism, hyperlexia, and attention deficit hyperactivity disorder (ADHD). Mammalian subjects amenable for treatment using the compositions and method of the invention additionally include, but are not limited to, human and other mammalian subjects suffering from related disorders including Landau-Kleffner Syndrome; Multi-systems disorder; anxiety disorders including, but not limited to, social phobia, generalized anxiety disorder, panic disorder, posttraumatic stress disorder, phobia, agoraphobia, obsessive-compulsive disorders; social deficit disorders including, but not limited to, paranoid personality disorder, schizotypal personality disorder, schizoid personality disorder, avoidant personality disorder, conduct disorder, borderline personality disorder, histrionic personality disorder; repetitive disorders including, but not limited to, impulse control and addiction disorders, and eating disorders such as bulimia, anorexia nervosa, binge eating disorder; cognitive deficit disorders including, but not limited to, dementia, Alzheimer's, Creutzfeld-Jakob disease, attention deficit disorder, attention deficit hyperactivity disorder, mild cognitive decline, and cognitive disorder not otherwise specified.

These and other subjects are effectively treated, prophylactically and/or therapeutically, by administering to the subject an effective amount of an oxytocin or oxytocin analog compound sufficient to prevent or reduce the occurrence or symptoms of autism spectrum disorders and related disorders. Therapeutically useful methods and formulations of the invention will effectively use oxytocin and oxytocin analogs in a variety of forms, as noted above, including any active, pharmaceutically acceptable salt of said compounds, as well as active isomers, enantiomers, polymorphs, solvates, hydrates, prodrugs and/or combinations thereof. Carbetocin is employed as an illustrative embodiment of the invention within the examples herein below.

Within additional aspects of the invention, combinatorial formulations and methods are provided comprising an effective amount of oxytocin or an oxytocin analog including carbetocin in combination with one or more secondary adjunctive agent(s) that is/are combinatorially formulated or coordinately administered with the oxytocin or oxytocin analog to yield an effective response in an individual suffering from autism spectrum disorders and related disorders. Exemplary combinatorial formulations and coordinate treatment methods in this context employ the oxytocin or oxytocin analog in combination with one or more additional, secondary or adjunctive therapeutic agents. The secondary or adjunctive therapeutic agents used in combination with, e.g., carbetocin, in these embodiments may possess direct or indirect anxiolytic activity alone or in combination with, e.g., carbetocin. The secondary or adjunctive therapeutic agents used in combination with, e.g., carbetocin, in these embodiments may possess direct or indirect antipsychotic activity alone or in combination with, e.g., carbetocin. The secondary or adjunctive therapeutic agents used in combination with, e.g., carbetocin, in these embodiments may possess direct or indirect anti-convulsant activity alone or in combination with, e.g., carbetocin. The secondary or adjunctive therapeutic agents used in combination with, e.g., carbetocin, in these embodiments may possess direct or indirect anti-viral activity alone or in combination with, e.g., carbetocin. Useful adjunctive therapeutic agents in these combinatorial formulations and coordinate treatment methods include, for example, serotonin reuptake inhibitors, selective serotonin reuptake inhibitors including, but not limited to, fluoxetine, fluvoxamine, sertraline, clomipramin; antipsychotic medications including, but not limited to, haloperidol, thioridazine, fluphenazine, chlorpromazine, risperidone, olanzapine, ziprasidone; anti-convulsants, including, but not limited to, carbamazepine, lamotrigine, topiramate, valproic acid, stimulant medications including, but not limited to, methylphenidate, α2-adrenergic agonists, amantadine, and clonidine; antidepressants including, but not limited to, naltrexone, lithium, and benzodiazepines; anti-virals, including, but not limited to valtrex; secretin; axiolytics including, but not limited to buspirone; immunotherapy. Additional adjunctive therapeutic agents include vitamins including but not limited to, B-vitamins (B6, B12, thiamin), vitamin A, and essential fatty acids. Adjunctive therapies may also include behavioral modification and changes in diet such as a gluten-casein free diet.

The forgoing objects and additional objects, features, aspects and advantages of the instant invention will become apparent from the following detailed description.

### BRIEF DESCRIPTION OF THE DRAWINGS

FIG. 1 is a graph depicting the percentage of permeation of formulations 13-44.

FIG. 2 is a graph depicting cytotoxicity according to an apical LDH assay of formulations 13-44.

### DETAILED DESCRIPTION OF THE INVENTION

The instant invention provides novel methods and compositions for preventing and/or treating psychiatric and neurological disorders including autism spectrum disorders, related disorders and symptoms of such disorders in mammalian subjects. In various embodiments, the present invention uses oxytocin and oxytocin analogs including carbetocin to treat such psychiatric and neurological disorder.

As used herein, the term “analog” or “agonist” refers to any molecule that demonstrates activity similar to that of the parent molecule. Such a molecule may be a synthetic analog, fragment, pharmaceutically acceptable salt, or endogenous biological molecule capable of similar activity to the parent compound.

Formulations for use in treating and preventing autism spectrum disorders, related disorders and symptoms of such disorders employ oxytocin or an oxytocin analog such as carbetocin, including all active pharmaceutically acceptable compounds of this description as well as various foreseen and readily provided complexes, derivatives, salts, solvates, isomers, enantiomers, polymorphs, and prodrugs of these compounds, and combinations thereof. Exemplary analogs for use within the invention include, as illustrative embodiments, 4-threonine-1-hydroxy-deaminooxytocin, 9-deamidooxytocin, an analog of oxytocin containing a glycine residue in place of the glycinamide residue; 7-D-proline-oxytocin and its deamino analog; (2,4-diisoleucine)-oxytocin, an analog of oxytocin with natriuretic and diuretic activities; deamino oxytocin analog; a long-acting oxytocin (OT) analog, 1-deamino-1-monocarba-E12-[Tyr(OMe)]-OT(dCOMOT); carbetocin, (1-butanoic acid-2-(O-methyl-L-tyrosine)-1-carbaoxytocin, or, alternatively, deamino-1 monocarba-(2-O-methyltyrosine)-oxytocin [d(COMOT)]); [Thr4-Gly7]-oxytocin (TG-OT); oxypressin; Ile-conopressin; atosiban; deamino-6-carba-oxytoxin (dC60), d[Lys(8)(5/6C-Fluorescein)]VT, d[Thr(4), Lys(8)(5/6C-Fluorescein)]VT, [HO(1)][Lys(8)(5/6C-Fluorescein)]VT, [HO(1)][Thr(4), Lys(8)(5/6CFluorescein)]VT, d[Om(8)(5/6C-Fluorescein)]VT, d[Thr(4), Om(8)(5/6C-Fluorescein)]VT, [HO(1)][Om(8)(5/6C-Fluorescein)]VT, [HO(1)][Thr(4), Om(8)(5/6C-Fluorescein)]VT, desmopressin, and 1-deamino-oxytocin in which the disulfide bridge between residues 1 and 6 is replaced by a thioether.

Within the formulations and methods, oxytocin or an oxytocin analog as disclosed herein is effectively used to treat autism spectrum disorders, related disorders and symptoms of such disorders in mammalian subjects suffering from autism spectrum disorders and/or related disorders and symptoms of such disorders including social withdrawal, eye contact avoidance, repetitive behaviors, anxiety, attention deficit, hyperactivity, depression, loss of speech, verbal communication difficulties, aversion to touch, visual difficulties, comprehension difficulties, and sound and light sensitivity.

A broad range of mammalian subjects, including human subjects, are amenable for treatment using the formulations and methods of the invention. These subjects include, but are not limited to, human and other mammalian subjects suffering from a psychiatric or neurological disorder including autism spectrum disorders such as autism, Asperger's syndrome, pervasive developmental disorder not otherwise specified, Rett's disorder, childhood disintegrative disorder, semantic pragmatic communication disorder, non-verbal learning disabilities, high functioning autism, hyperlexia, and ADHD. Mammalian subjects amenable for treatment using the compositions and methods of the invention additionally include, but are not limited to, human and other mammalian subjects suffering from related disorders including Landau-Kleffner Syndrome; multi-systems disorder; anxiety disorders including, but not limited to, social phobia, generalized anxiety disorder, panic disorder, posttraumatic stress disorder, phobia, agoraphobia, obsessive-compulsive disorders; social deficit disorders including, but not limited to, paranoid personality disorder, schizotypal personality disorder, schizoid personality disorder, avoidant personality disorder, conduct disorder, borderline personality disorder, histrionic personality disorder; repetitive disorders including, but not limited to, impulse control and addiction disorders, and eating disorders such as bulimia, anorexia nervosa, binge eating disorder; cognitive deficit disorders including, but not limited to, dementia, Alzheimer's, Creutzfeld-Jakob disease, attention deficit disorder, attention deficit hyperactivity disorder, mild cognitive decline, and cognitive disorder not otherwise specified.

Within the methods and compositions of the invention, one or more oxytocin analogs as disclosed herein is/are effectively formulated or administered as a psychiatric or neurologic treating agent effective for treating autism spectrum disorders, related disorders and symptoms of such disorders. In exemplary embodiments, carbetocin is used for illustrative purposes alone or in combination with one or more adjunctive therapeutic agent(s). The present disclosure further provides additional, pharmaceutically acceptable oxytocin analogs in the form of a native or synthetic compound, including complexes, derivatives, salts, solvates, isomers, enantiomers, polymorphs, and prodrugs of the compounds disclosed herein, and combinations thereof, which are effective as autism spectrum disorders and related disorder treating agents within the methods and compositions of the invention.

Autism spectrum disorders are defined by specific behaviors that can range from mild to severe. Symptoms include deficits in social interaction, verbal and nonverbal communication and repetitive behaviors and interests. The development of impairments in autistic persons is varied and characteristically uneven, resulting in good skills in some areas and poor skills in others. Echolalia is a common feature of language impairment that, when present, may cause language skills to appear better than they really are. There may also be deficiencies in symbolic thinking, stereotypic behaviors (e.g., repetitive nonproductive movements of hands and fingers, rocking, meaningless vocalizations), self-stimulation, self-injury behaviors, and seizures. No single cause has been identified for the development of autism though genetic origins are suggested by studies of twins and a higher incidence of recurrence among siblings. In addition, an increased frequency of autism is found in individuals with genetic conditions such as fragile X syndrome and tuberous sclerosis. Possible contributing factors in the development of autism include infections, errors in metabolism, immunology, lead poisoning, and fetal alcohol syndrome. The compositions and methods of the present invention are effective in the treatment of all types of autism spectrum disorders, regardless of cause.

Oxytocin is a mammalian hormone secreted by the pituitary gland that acts as a neurotransmitter and is known to stimulate uterine contractions and milk let down. It is a peptide of nine amino acids in the sequence cysteine-tyrosine-isoleucine-glutamine-asparagine -cysteine-proline-leucine-glycine (CYIQNCPLG). Based on a review of evidence from animal studies demonstrating that the nonapeptides, oxytocin and vasopressin, have unique effects on the normal expression of species-typical social behavior, communication and rituals, it was proposed that oxytocin or vasopressin neurotransmission may account for several features associated with autism. (Insel, et al., Biol. Psychiatry 45:145-157, 1999). A study on autistic children reported that such children had significantly lower levels of plasma oxytocin than normal children. Elevated oxytocin levels were associated with higher scores on social and developmental tests in non-autistic children, but associated with lower scores in autistic children, suggesting that altered oxytocin levels may be associated with autism in children (Modahl, et al., Biol. Psychiatric 43:270-277, 1998). Elevated levels of oxytocin have additionally been implicated in certain obsessive-compulsive behaviors such as excessive worrying, sexual compulsions and/or compulsive washing and cleaning. (Leckman, et al., Psychoneuroendocrinology 19:723-749, 1994; Leckman, et al., Arch Gen Psychiatry 51:782-92, 1994). Elevated levels of oxytocin have also been implicated in Prader-Willi syndrome, a genetic disorder associated with mental retardation, appetite dysregulation and a risk of developing obsessive compulsive disorder (Martin, et al., Biol. Psychiatric 44:1349-1352, 1998).

A number of oxytocin analogs have been evaluated as possible substitute agents for inducing uterine contraction and milk let-down in mammalian patients with the goal of minimizing oxytocin's side effects. One such analog, carbetocin (1-butanoic acid-2-(O-methyl-L-tyrosine)-1-carbaoxytocin, or, alternatively, deamino-1 monocarba-(2-O-methyltyrosine)-oxytocin [d(COMOT)]) is a long-acting synthetic oxytocin analog which exhibits both uterotonic and milk let-down inducing activities (Alke, et al., Acta Endocrinol. 115:155-160, 1987; Norstrom, et al., Acta Endocrinol. 122:566-568, 1990; Hunter, et al., Clin. Pharmacol. Ther. 52:60-67, 1992; Silcox, et al., Obstet. Gynecol. 82:456-459, 1993; Vilhardt, et al., Pharmacol. Toxicol. 81:147-150, 1997; Boucher, et al., J. Perinatology 18:202-207, 1998). The half-life of carbetocin is reportedly 4 to 10 times longer than that of oxytocin, which is reflected in substantial prolongation of the uterotonic and milk let-down inducing activities of this analog. This apparent increase in metabolic stability is attributed to N-terminal desamination and replacement of a 1-6 disulfide bridge by a methylene group in carbetocin, which modifications are thought to protect this analog from aminopeptidase and disulfidase cleavage (Hunter, et al., Clin. Pharmacol. Ther. 52:60-67, 1992). The methods and compositions of the present invention comprise the use of oxytocin and oxytocin analogs in novel formulations for the treatment of neurological and psychiatric disorders including autism spectrum disorders and related disorders such as obsessive compulsive disorders.

The compositions and methods of the instant invention represented by carbetocin are effective for treating or preventing psychiatric and neurological disorders in mammals. In particular, the compositions and methods of the invention can be administered to mammalian subjects to measurably alleviate or prevent one or more symptoms of an autism spectrum disorder or a related condition, selected from symptoms including, but not limited to, social withdrawal, eye contact avoidance, repetitive behaviors, anxiety, attention deficit, hyperactivity, depression, loss of speech, verbal communication difficulties, aversion to touch, visual difficulties, comprehension difficulties, and sound and light sensitivity.

Compositions comprising carbetocin or other oxytocin analogs for the treatment of autism spectrum disorders, related disorders and symptoms of such disorders, comprise an amount of carbetocin or other oxytocin analog which is effective for prophylaxis and/or treatment of autism spectrum disorders, related disorders and symptoms of such disorders in a mammalian subject. Typically an effective amount of the carbetocin or other oxytocin analog will comprise an amount of the active compound which is therapeutically effective, in a single or multiple dosage form, over a specified period of therapeutic intervention, to measurably alleviate one or more symptoms of autism spectrum disorders and/or related disorders in the subject. Within exemplary embodiments, these compositions are effective within in vivo treatment methods to alleviate autism spectrum disorders and related disorders.

Autism spectrum and related disorder treating compositions of the invention typically comprise an effective amount or unit dosage of oxytocin or an oxytocin analog which may be formulated with one or more pharmaceuctically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. Exemplary excipients include solubilizers surfactants and chelators, for example formulations may include, methyl-β-cyclodextrin (Me-β-CD), edetate disodium (EDTA), arginine, sorbitol, NaCl, methylparaben sodium (MP), propylparaben sodium (PP), chlorobutanol (CB), benzyl alcohol, zinc chloride, ethyl alcohol, didecanoyl L-α-phosphatidylcholine (DDPC), polysorbate, lactose, citrate, tartrate, acetate, and or phosphate. Effective amounts of oxytocin or an oxytocin analog such as carbetocin for the treatment of neurological and psychiatric disorders (e.g., a unit dose comprising an effective concentration/amount of carbetocin, or of a selected pharmaceutically acceptable salt, isomer, enantiomer, solvate, polymorph and/or prodrug of carbetocin) will be readily determined by those of ordinary skill in the art, depending on clinical and patient-specific factors. Suitable effective unit dosage amounts of the active compounds for administration to mammalian subjects, including humans, may range from 10 to 1500 μg, 20 to 1000 μg, 25 to 750 μg, 50 to 500 μg, or 150 to 500 μg, 10 to 1500 mg, 20 to 1000 mg, 25 to 750 mg, 50 to 500 mg, or 150 to 500 mg. In certain embodiments, the effective dosage of oxytocin or an oxytocin analog may be selected within narrower ranges of, for example, 10 to 25 μg, 30-50 μg, 75 to 100 μg, 100 to 250 μg, or 250 to 500 μg, 10 to 25 mg, 30-50 mg, 75 to 100 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In one exemplary embodiment, dosages of 10 to 25 mg, 30-50 mg, 75 to 100 mg, 100 to 250 mg, or 250 to 500 mg, are administered one, two, three, four, or five times per day. In more detailed embodiments, dosages of 50-75 mg, 100-200 mg, 250-400 mg, or 400-600 mg are administered once or twice daily. In alternate embodiments, dosages are calculated based on body weight, and may be administered, for example, in amounts from about 0.5 mg/kg to about 100 mg/kg per day, 1 mg/kg to about 75 mg/kg per day, 1 mg/kg to about 50 mg/kg per day, 2 mg/kg to about 50 mg/kg per day, 2 mg/kg to about 30 mg/kg per day or 3 mg/kg to about 30 mg/kg per day.

The amount, timing and mode of delivery of compositions of the invention comprising an effective amount of carbetocin or other oxytocin analog will routinely be adjusted on an individual basis, depending on such factors as weight, age, gender, and condition of the individual, the acuteness of the autism spectrum disorders, related disorders and/or symptoms of such disorders, whether the administration is prophylactic or therapeutic, and on the basis of other factors known to effect drug delivery, absorption, pharmacokinetics, including half-life, and efficacy.

An effective dose or multi-dose treatment regimen for the instant formulations will ordinarily be selected to approximate a minimal dosing regimen that is necessary and sufficient to substantially prevent or alleviate autism spectrum disorders, related disorders and/or symptoms of such disorders in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years.

Various assays and model systems can be readily employed to determine the therapeutic effectiveness of oxytocin or an oxytocin analog in the treatment of autism spectrum disorders and related disorders. The effectiveness of the compositions for these and related conditions can be routinely demonstrated according to a variety of methods, including, for example, by measuring markers such as those measured in the Checklist of Autism in Toddlers (CHAT), the modified Checklist for Autism in Toddlers (M-CHAT), the Screening Tool for Autism in Two-Year-Olds (STAT), the Social Communication Questionnaire (SCQ), the Autism Spectrum Screening Questionnaire (ASSQ), the Australian Scale for Asperger's Syndrome, the Childhood Asperger Syndrome Test (CAST), the Autism Diagnosis Interview-Revised (ADI-R), the Autism Diagnostic Observation Schedule (ADOS-G), the Childhood Autism Rating Scale (CARS), audiologic hearing evaluation, Administered PTSD Scale, the Eysenck Personality Inventory, the Hamilton Anxiety Scale, or in various animal models such as the well-known Vogel (thirsty rat conflict) test, or the elevated plus maze test. Effective amounts of a compound of oxytocin or an oxytocin analog will measurably prevent, decrease the severity of, or delay the onset or duration of, one or more of the foregoing autism spectrum disorders, related disorders of symptoms of such disorders in a mammalian subject.

Administration of an effective amount of oxytocin or an oxytocin analog such as carbetocin to a subject presenting with one or more of the foregoing symptom(s) will detectably decrease, eliminate, or prevent the subject symptom(s). In exemplary embodiments, administration of a compound of carbetocin to a suitable test subject will yield a reduction in one or more target symptom(s) associated with a neurological or psychiatric disorder by at least 10%, 20%, 30%, 50% or greater, up to a 75-90%, or 95% or greater, reduction in the one or more target symptom(s) or disorders, compared to placebo-treated or other suitable control subjects. Comparable levels of efficacy are contemplated for the entire range of neurological and psychiatric disorders identified herein for treatment or prevention using the compositions and methods of the invention. Within additional aspects of the invention, combinatorial formulations and coordinate administration methods are provided which employ an effective amount of oxytocin or an oxytocin analog such as carbetocin and one or more secondary or adjunctive agent(s) that is/are combinatorially formulated or coordinately administered with the oxytocin or oxytocin analog to yield a combined, multi-active agent or coordinate treatment method. Exemplary combinatorial formulations and coordinate treatment methods in this context employ the oxytocin or oxytocin analog in combination with one or more secondary psychiatric or neurological agent(s) or with one or more adjuntive therapeutic agent(s) that is/are useful for treatment or prophylaxis of the targeted disease, condition and/or symptom(s) in the selected combinatorial formulation or coordinate treatment regimen. For most combinatorial formulations and coordinate treatment methods of the invention, oxytocin or a related analog is formulated, or coordinately administered, in combination with one or more secondary or adjunctive therapeutic agent(s) to yield a combined formulation or coordinate treatment method that is combinatorially effective or coordinately useful to treat autism spectrum disorders or related disorders and/or one or more symptom(s) of such disorders. Exemplary combinational formulations and coordinate treatment methods in this context employ oxytocin or an oxytocin analog in combination with one or more secondary or adjunctive therapeutic agents selected from, e.g., serotonin reuptake inhibitors, selective serotonin reuptake inhibitors including, but not limited to, fluoxetine, fluvoxamine, sertraline, clomipramin; antipsychotic medications including, but not limited to, haloperidol, thioridazine, fluphenazine, chlorpromazine, risperidone, olanzapine, and ziprasidone; anti-convulsants, including, but not limited to, carbamazepine, lamotrigine, topiramate, and valproic acid, stimulant medications including, but not limited to, methylphenidate, α2-adrenergic agonists, amantadine, and clonidine; antidepressants including, but not limited to monoamine oxidase inhibitors, including phenelzine and isocarboxazide, tricyclic antidepressants, including amitriptaline, clomipramine, desipramine, and nortriptyline, atypical antidepressants (non-SSRIs), including Bupropion (Wellbutrin), Velafaxine (Effexor), and SSRIs such as Citalopram, Fluoxetine, Fluvoxamine, Paroxetine, and Sertraline; axiolytics including, but not limited to benzodiazepine and buspirone. Additional adjunctive therapeutic agents include vitamins including but not limited to, B-vitamins (B6, B12, thiamin), vitamin A, and essential fatty acids. Adjunctive therapies may include behavioral modification and changes in diet such as a gluten-casein free diet.

Within additional aspects of the invention, combinatorial formulations and coordinate administration methods are provided which employ an effective amount of one or more compounds of oxytocin or an oxytocin analog, and one or more additional active agent(s) that is/are combinatorially formulated or coordinately administered with the oxytocin or oxytocin analog yielding an effective formulation or method to treat autism spectrum disorders, related disorders and symptoms of such disorders, and/or to alleviate or prevent one or more symptom(s) of a neurological or psychiatric disorder in a mammalian subject. Exemplary combinatorial formulations and coordinate treatment methods in this context employ oxytocin or an oxytocin analog in combination with one or more additional or adjunctive anxiolytic, antidepressant, anticonvulsant, nootropic, antipsychotic, stimulant, anti-viral, immunotherapeutic, anesthetic, hypnotic or muscle relaxant agent(s). In additional combinatorial formulations and coordinate treatment methods, oxytocin or an oxytocin analog is formulated or co-administered in combination with one or more secondary therapeutic agents used to treat symptoms which may accompany the psychiatric or neurological conditions listed above.

To practice the coordinate administration methods of the invention, oxytocin or an oxytocin analog is administered, simultaneously or sequentially, in a coordinate treatment protocol with one or more of the secondary or adjunctive therapeutic agents contemplated herein. The coordinate administration may be done simultaneously, or sequentially in either order, and there may be a time period while only one or both (or all) active therapeutic agents, individually and/or collectively, exert their biological activities. A distinguishing aspect of all such coordinate treatment methods is that the oxytocin or oxytocin analog such as carbetocin exerts at least some detectable therapeutic activity, and/or elicits a favorable clinical response, which may or may not be in conjunction with a secondary clinical response provided by the secondary therapeutic agent. Often, the coordinate administration of oxytocin or an oxytocin analog such as carbetocin with a secondary therapeutic agent as contemplated herein will yield an enhanced therapeutic response beyond the therapeutic response elicited by either or both the oxytocin analog and/or secondary therapeutic agent alone.

Within exemplary embodiments, oxytocin, or an oxytocin analog will be coordinately administered (simultaneously or sequentially, in combined or separate formulation(s)), with one or more secondary agents or other indicated therapeutic agents, e.g., selected from, for example, serotonin reuptake inhibitors, selective serotonin reuptake inhibitors including, but not limited to, fluoxetine, fluvoxamine, sertraline, clomipramin; antipsychotic medications including, but not limited to, haloperidol, thioridazine, fluphenazine, chlorpromazine, risperidone, olanzapine, ziprasidone; anti-convulsants, including, but not limited to, carbamazepine, lamotrigine, topiramate, valproic acid, stimulant medications including, but not limited to, methylphenidate, α2-adrenergic agonists, amantadine, and clonidine; antidepressants including, but not limited to, naltrexone, lithium, and benzodiazepines; anti-virals, including, but not limited to valtrex; secretin; axiolytics including, but not limited to buspirone; immunotherapy. Additional adjunctive therapeutic agents include vitamins including but not limited to, B-vitamins (B6, B12, thiamin), vitamin A, and essential fatty acids. Adjunctive therapies may include behavioral modification and changes in diet such as a gluten-casein free diet.

In certain embodiments, the invention provides combinatorial neurological and psychiatric treating formulations comprising oxytocin and one or more adjunctive agent(s) having effective activity for the treatment of autism spectrum disorders and related disorders. Within such combinatorial formulations, oxytocin and oxytocin analogs and the adjunctive agent(s) will be present in a combined formulation in effective amounts, alone or in combination. In exemplary embodiments, oxytocin or an oxytocin analog such as carbetocin will be present in an effective amount. Alternatively, the combinatorial formulation may comprise one or both of the active agents in sub-therapeutic singular dosage amount(s), wherein the combinatorial formulation comprising both agents features a combined dosage of both agents that is collectively effective in eliciting a desired response. Thus, one or both of the oxytocin or oxytocin analog and additional agents may be present in the formulation, or administered in a coordinate administration protocol, at a sub-therapeutic dose, but collectively in the formulation or method they elicit a detectable response in the subject.

As noted above, in all of the various embodiments of the invention contemplated herein, the formulations may employ oxytocin or an oxytocin analog in any of a variety of forms, including any one or combination of the subject compound's pharmaceutically acceptable salts, isomers, enantiomers, polymorphs, solvates, hydrates, and/or prodrugs. In exemplary embodiments of the invention, berberine is employed within the therapeutic formulations and methods for illustrative purposes.

The pharmaceutical compositions of the present invention may be administered by any means that achieves their intended therapeutic or prophylactic purpose. Suitable routes of administration include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Pharmaceutical dosage forms of the oxytocin analog of the present invention include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, tonicifiers, effervescent agents and other conventional excipients and additives.

In one embodiment, carbetocin or other oxytocin analog will be combined with a solubilizer, surfactant, tonicifiers, preservatives, buffers, and chelator. Such excipients include, but are not limited to, methyl-β-cyclodextrin (Me-β-CD), edetate disodium (EDTA), arginine, sorbitol, NaCl, methylparaben sodium (MP), propylparaben sodium (PP), chlorobutanol (CB), benzyl alcohol, zinc chloride, ethyl alcohol, didecanoyl L-α-phosphatidylcholine (DDPC), polysorbate, lactose, citrate, tartrate, acetate, and or phosphate. Exemplary surfactants additionally include, but are not limited to, DMSO, Tween™ (including but not limited to, Tween 80 (polysorbate 80) and Tween 20 (polysorbate 20), Pluronics™ and other pluronic acids, including but not limited to, pluronic acid F68 (poloxamer 188), PEG; polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. In another embodiment, the composition contains a solubilizer in combination with carbetocin or other oxytocin analog. In a further embodiment, the composition contains a surfactant in combination with carbetocin or other oxytocin analog. In yet another embodiment, the composition contains a chelator in combination with carbetocin or other oxytocin analog. Compositions of the present invention may further contain combinations of solubilizers, surfactants and chelators. For example the composition of the present invention may contain methyl-β-cyclodextrin and edetate disodium in combination with carbetocin or other oxytocin analog.

The compositions of the invention for treating neurological and psychiatric disorders including autism spectrum disorders and related disorders can thus include any one or combination of the following: a pharmaceutically acceptable carrier or excipient; other medicinal agent(s); pharmaceutical agent(s); adjuvants; buffers; solubilizers, surfactants, chelators, preservatives; diluents; and various other pharmaceutical additives and agents known to those skilled in the art. These additional formulation additives and agents will often be biologically inactive and can be administered to patients without causing deleterious side effects or interactions with the active agent.

If desired, the oxytocin analogs of the invention can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps.

Oxytocin or oxytocin analog compositions of the invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute attacks of panic disorder.

Additional oxytocin or oxytocin analog compositions of the invention can be prepared and administered in any of a variety of inhalation or nasal delivery forms known in the art. Devices capable of depositing aerosolized oxytocin formulations in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of seizures or panic disorder. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of oxytocin or oxytocin analogs and any additional active or inactive ingredient(s).

Intranasal delivery permits the passage of such a compound to the blood stream directly after administering an effective amount of the compound to the nose, without requiring the product to be deposited in the lung. In addition, intranasal delivery can achieve direct, or enhanced, delivery of the active compound to the central nervous system. In these and other embodiments, intranasal administration of the compounds of the invention may be advantageous for treating sudden onset anxiety disorders, such as panic disorder. Typically, the individual suffering from generalized anxiety disorder and prone to attacks of panic disorder is able to sense when such an attack is imminent. At such times, it is particularly desirable to be able to administer compounds of the invention in a form that is convenient even in a public setting, and that yields rapid absorption and central nervous system delivery.

For intranasal and pulmonary administration, a liquid aerosol formulation will often contain an active compound of the invention combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of oxytocin or oxytocin analogs for treating neurological and psychiatric disorders including autism spectrum disorders, related disorders and symptoms of such disorders.

Topical compositions may comprise oxytocin or oxytocin analogs and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise oxytocin or oxytocin analogs dissolved or dispersed in a portion of a water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours. A once-daily transdermal patch is particularly useful for a patient suffering from generalized anxiety disorder.

Yet additional oxytocin or oxytocin analogs are provided for parenteral administration, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Oxytocin or oxytocin analogs may also include polymers for extended release following parenteral administration. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In more detailed embodiments, oxytocin or oxytocin analogs may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macro emulsions.

As noted above, in certain embodiments the methods and compositions of the invention may employ pharmaceutically acceptable salts, e.g., acid addition or base salts of the above-described oxytocin or oxytocin analog. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts. Suitable base salts are formed from bases that form non-toxic salts, for example aluminum, calcium, lithium, magnesium, potassium, sodium, zinc and diethanolamine salts.

The pharmaceutical agents of the invention may be administered parenterally, e.g., intravenously, intramuscularly, subcutaneously or intraperitoneally. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such polymeric materials are well known to those of ordinary skill in the pharmaceutical compounding arts. Parenteral preparations typically contain buffering agents and preservatives, and may be lyophilized to be re-constituted at the time of administration.

The invention disclosed herein will also be understood to encompass methods and compositions comprising oxytocin or oxytocin analogs using in vivo metabolic products of the said compounds (either generated in vivo after administration of the subject precursor compound, or directly administered in the form of the metabolic product itself). Such products may result for example from the oxidation, reduction, hydrolysis, amidation, esterification, glycosylation and the like of the administered compound, primarily due to enzymatic processes. Accordingly, the invention includes methods and compositions of the invention employing compounds produced by a process comprising contacting a berberine related or derivative compound of oxytocin or oxytocin analogs with a mammalian subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein will also be understood to encompass diagnostic compositions for diagnosing the risk level, presence, severity, or treatment indicia of, or otherwise managing oxytocin or oxytocin analogs in a mammalian subject, comprising contacting a labeled (e.g., isotopically labeled, fluorescent labeled or otherwise labeled to permit detection of the labeled compound using conventional methods) oxytocin or oxytocin analog to a mammalian subject (e.g., to a cell, tissue, organ, or individual) at risk or presenting with one or more symptom(s) of autism spectrum disorders or related disorders, and thereafter detecting the presence, location, metabolism, and/or binding state of the labeled compound using any of a broad array of known assays and labeling/detection methods.

In exemplary embodiments, oxytocin or an oxytocin analog such as carbetocin is isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. The isotopically-labeled compound is then administered to an individual or other subject and subsequently detected as described above, yielding useful diagnostic and/or therapeutic management data, according to conventional techniques.

### EXAMPLES

### Example I

### Permeation of Carbetocin Formulations

Permeation studies on varying formulations of carbetocin were completed using tracheal/bronchial epithelial cell membrane inserts. Samples were evaluated for appearance, color, clarity, pH, osmolality, cell viability using an MTT assay, cytotoxicity using an LDH assay, and transepithelial resistance and permeation.

Samples were prepared according to the formulas in Table 1.

TABLE 1
Sample Composition of Carbetocin Formulations

| | | Me-β- | | | Polysorbate | NaCl | | Lac- | Chloro- | | | | | |
|---|---|---|---|---|---|---|---|---|---|---|---|---|---|---|
| | carbetocin | CD | DDPC | EDTA | 80 | (mg/ | Sorbitol | tose | butanol | MP/PP | ZnCl2 | EtOh | | |
| # | (mg/ml) | (mg/ml) | (mg/ml) | (mg/ml) | (mg/ml) | mL) | (mM) | (mM) | (mg/mL) | (mg/ml) | (mM) | mg/ml | Buffer | pH |
| 1 | 10 | 45 | 1 | 1 | 0 | 0 | 100 | 25 | 0 | — | — | — | 10 mM | 4.00 |
| | | | | | | | | | | | | | arginine | |
| 2 | 10 | 30 | 1.7 | 2 | 0 | 4 | 0 | 0 | 0 | — | — | — | — | 4.00 |
| 3 | 10 | 0 | 0 | 2.5 | 1 | 0 | 131 | 0 | 5 | — | — | — | — | 4.00 |
| 4 | 10 | 45 | 0 | 1 | 10 | 3.5 | 0 | 0 | 0 | — | — | — | 10 mM | 5.00 |
| | | | | | | | | | | | | | arginine | |
| 5 | 10 | 80 | 0 | 5 | 0 | 1.5 | 0 | 0 | 0 | — | — | — | 2.8 mM | 5.25 |
| | | | | | | | | | | | | | arginine | |
| | | | | | | | | | | | | | 10 mM | |
| | | | | | | | | | | | | | acetate | |
| 6 | 10 | 0 | 0 | 0 | 0 | 8.75 | 0 | 0 | 0 | — | — | — | 10 mM | 5.00 |
| | | | | | | | | | | | | | acetate | |
| 7 | 10 | 0 | — | 2.5 | 1 | 0 | 131 | — | 5 | — | — | — | 0 | 4.00 |
| 8 | 10 | 0 | — | 2.5 | 0 | 0 | 131 | — | 5 | — | — | — | 0 | 4.00 |
| 9 | 10 | 0 | — | 5 | 0 | 0 | 90 | — | 5 | — | — | — | 10 mM | 4.00 |
| | | | | | | | | | | | | | arginin | |
| 10 | 10 | 80 | — | 5 | 0 | 1.5 | 0 | — | 0 | — | — | — | 2.8 mM | 5.25 |
| | | | | | | | | | | | | | arginine | |
| | | | | | | | | | | | | | 10 mM | |
| | | | | | | | | | | | | | acetate | |
| 11 | 10 | 40 | — | 5 | 0 | 1.8 | 0 | — | 5 | — | — | — | 10 | 5.25 |
| 12 | 10 | 0 | — | 0 | 0 | 8.75 | 0 | — | 0 | — | — | — | 10 mM | 5.00 |
| | | | | | | | | | | | | | acetate | |
| 13 | 2 | 0 | 0 | 2.5 | — | 0 | 131 | | 0 | — | — | — | 0 | 3.7 +/− 0.2 |
| 14 | 2 | 20 | 0 | 5 | — | | 0 | | 0 | — | — | — | 0 | 3.7 +/− 0.2 |
| 15 | 2 | 40 | 0.5 | 5 | — | | 0 | | 0 | — | — | — | 0 | 3.7 +/− 0.2 |
| 16 | 2 | 0 | 0 | 5 | — | | 0 | | 0 | — | — | — | 0 | 3.7 +/− 0.2 |
| 17 | 2 | 10 | 0 | 5 | — | | 0 | | 0 | — | — | — | 0 | 3.7 +/− 0.2 |
| 18 | 2 | 0 | 0 | 2.5 | — | 0 | 131 | | 5 | — | — | — | 0 | 3.7 +/− 0.2 |
| 19 | 2 | 0 | 0 | 2.5 | — | | 0 | | 0 | — | — | — | 0 | 3.7 +/− 0.2 |
| 20 | 2 | 40 | 1 | 5 | — | | 0 | | 0 | — | — | — | 0 | 3.7 +/− 0.2 |
| 21 | 2 | 40 | 0.25 | 2.5 | — | | 0 | | 0 | — | — | — | 0 | 3.7 +/− 0.2 |
| 22 | 2 | 10 | 0 | 0 | — | | 0 | | 0 | — | — | — | 0 | 3.7 +/− 0.2 |
| 23 | 2 | 40 | 1 | 0 | — | | 0 | | 0 | — | — | — | 0 | 3.7 +/− 0.2 |
| 24 | 2 | 40 | 0 | 5 | — | 40 | 0 | | 0 | — | — | — | 10 mM | 3.7 +/− 0.2 |
| | | | | | | | | | | | | | arginine | |
| 25 | 2 | 30 | 0 | 2.5 | — | | 0 | | 0 | — | — | — | 0 | 3.7 +/− 0.2 |
| 26 | 2 | 20 | 0.5 | 0 | — | | 0 | | 0 | — | — | — | 0 | 3.7 +/− 0.2 |
| 27 | 2 | 40 | 0 | 0 | — | | 0 | | 0 | — | — | — | 0 | 3.7 +/− 0.2 |
| 28 | 2 | 30 | 0.5 | 2.5 | — | | 0 | | 0 | — | — | — | 0 | 3.7 +/− 0.2 |
| 29 | 2 | 40 | 1 | 2.5 | — | | 0 | | 0 | — | — | — | 0 | 3.7 +/− 0.2 |
| 30 | 2 | 40 | 0 | 5 | — | | 0 | | 0 | — | — | — | 0 | 3.7 +/− 0.2 |
| 31 | 2 | 40 | 0 | 5 | — | 40 | 0 | | 5 | — | — | — | 10 | 3.7 +/− 0.2 |
| 32 | 2 | 20 | 0.25 | 5 | — | | 0 | | 0 | — | — | — | 0 | 3.7 +/− 0.2 |
| 33 | 2 | 0 | 0 | 0 | — | | 0 | | 0 | — | — | — | 0 | 3.7 +/− 0.2 |
| 34 | 2 | 20 | 0 | 0 | — | | 0 | | 0 | — | — | — | 0 | 3.7 +/− 0.2 |
| 35 | 2 | 10 | 0.25 | 3.75 | — | | 0 | | 0 | — | — | — | 0 | 3.7 +/− 0.2 |
| 36 | 2 | 10 | 0 | 2.5 | — | | 0 | | 0 | — | — | — | 0 | 3.7 +/− 0.2 |
| 37 | 2 | 30 | 0 | 5 | — | | 0 | | 0 | — | — | — | 0 | 3.7 +/− 0.2 |
| 38 | 2 | 20 | 0.5 | 5 | — | | 0 | | 0 | — | — | — | 0 | 3.7 +/− 0.2 |
| 39 | 2 | 40 | 0.5 | 0 | — | | 0 | | 0 | — | — | — | 0 | 3.7 +/− 0.2 |
| 40 | 2 | 40 | 0 | 2.5 | — | | 0 | | 0 | — | — | — | 0 | 3.7 +/− 0.2 |
| 41 | 2 | 5 | 0 | 5 | — | | 0 | | 0 | — | — | — | 0 | 3.7 +/− 0.2 |
| 42 | 2 | 30 | 1 | 5 | — | | 0 | | 0 | — | — | — | 0 | 3.7 +/− 0.2 |
| 43 | 2 | 0 | 0 | 0 | — | 0 | 0 | | 0 | — | — | — | 0 | 3.7 +/− 0.2 |
| 44 | 2 | 0 | 0 | 0 | — | 0 | 0 | | 0 | — | — | — | 0 | 3.7 +/− 0.2 |
| 45 | 2 | 0 | — | 3.5 | — | 57 | 0 | | 5 | 0 | — | | 10 mM | 4.00 |
| | | | | | | | | | | | | | arginine | |
| 46 | 2 | 10 | — | 3.5 | — | 52 | 0 | | 5 | 0 | — | | 10 mM | 4.00 |
| | | | | | | | | | | | | | arginine | |
| 47 | 2 | 10 | — | 3.5 | — | 0 | 104 | | 5 | 0 | — | | 10 mM | 4.00 |
| | | | | | | | | | | | | | arginine | |
| 48 | 2 | 20 | — | 3.5 | — | 50 | 0 | | 5 | 0 | — | | 10 mM | 4.00 |
| | | | | | | | | | | | | | arginine | |
| 49 | 4 | 10 | — | 3.5 | — | 52 | 0 | | 5 | 0 | — | | 10 mM | 4.00 |
| | | | | | | | | | | | | | arginine | |
| 50 | 3 | 10 | — | 3.5 | — | 65 | 0 | | 0 | 0.33/0.17 | — | | 10 mM | 4.00 |
| | | | | | | | | | | | | | arginine | |
| 51 | 3 | 10 | — | 3.5 | — | 60 | 0 | | 2.5 | 0 | — | | 10 mM | 4.00 |
| | | | | | | | | | | | | | arginine | |
| 52 | 3 | 10 | — | 3.5 | — | 52 | 0 | | 5 | 0 | — | | 10 mM | 4.00 |
| | | | | | | | | | | | | | arginine | |
| 53 | 3 | 10 | — | 3.5 | — | 50 | 0 | | 5 | 0.33/0.17 | — | | 10 mM | 4.00 |
| | | | | | | | | | | | | | arginine | |
| 54 | 3 | 0 | — | 3.5 | — | 70 | 0 | | 0 | 0.33/0.17 | — | | 10 mM | 4.00 |
| | | | | | | | | | | | | | arginine | |
| 55 | 3 | 20 | — | 3.5 | — | 60 | 0 | | 0 | 0.33/0.17 | — | | 10 mM | 4.00 |
| | | | | | | | | | | | | | arginine | |
| 56 | 2 | 40 | — | 5 | — | 40 | 0 | | 5 | 0 | — | | 10 mM | 4.50 |
| | | | | | | | | | | | | | arginine | |
| 57 | 2 | 0 | — | 2.5 | — | 0 | 131 | | 5 | 0 | — | | 0 | 4.00 |
| 58 | 2 | 0 | — | 0 | — | | 0 | | 0 | 0 | — | | 10 mM | 7.00 |
| | | | | | | | | | | | | | arginine | |
| 59 | 0 | 0 | — | 0 | — | 0 | 0 | | 0 | 0 | — | | 0 | 0.00 |
| 60 | 0 | 0 | — | 0 | — | 0 | 0 | | 0 | 0 | — | | 0 | 0.00 |
| 61 | 2 | 10 | — | 0 | — | — | 0 | 60 | 0 | — | 2 | 1 | 5 mM | — |
| | | | | | | | | | | | | | arginine | |
| 62 | 2 | 10 | — | 2.5 | — | — | 0 | 50 | 0 | — | 2 | 1 | 5 mM | — |
| | | | | | | | | | | | | | arginine | |
| 63 | 2 | 10 | — | 2.5 | — | — | 0 | 40 | 0 | — | 2 | 1 | 15 mM | — |
| | | | | | | | | | | | | | arginine | |
| 64 | 2 | 20 | — | 3.75 | — | — | 0 | 65 | 0 | — | 0 | 0 | 10 mM | — |
| | | | | | | | | | | | | | arginine | |
| 65 | 2 | 0 | — | 3.75 | — | — | 0 | 25 | 0 | — | 4 | 2 | 0 | — |
| 66 | 2 | 0 | — | 0 | — | — | 0 | 45 | 0 | — | 0 | 2 | 0 | — |
| 67 | 2 | 0 | — | 0 | — | — | 0 | 85 | 0 | — | 4 | 0 | 0 | — |
| 68 | 2 | 20 | — | 3.75 | — | — | 0 | 10 | 0 | — | 4 | 2 | 10 mM | — |
| | | | | | | | | | | | | | arginine | |
| 69 | 2 | 0 | — | 0 | — | — | 0 | 30 | 0 | — | 4 | 2 | 10 mM | — |
| | | | | | | | | | | | | | arginine | |
| 70 | 2 | 10 | — | 4 | — | — | 0 | 45 | 0 | — | 0 | 1 | 5 mM | — |
| | | | | | | | | | | | | | arginine | |
| 71 | 2 | 0 | — | 3.75 | — | — | 0 | 70 | 0 | — | 4 | 0 | 10 mM | — |
| | | | | | | | | | | | | | arginine | |
| 72 | 2 | 20 | — | 0 | — | — | 0 | 80 | 0 | — | 4 | 0 | 10 mM | — |
| | | | | | | | | | | | | | arginine | |
| 73 | 2 | 10 | — | 3.5 | — | — | 0 | 45 | 0 | — | 0 | 1 | 5 mM | — |
| | | | | | | | | | | | | | arginine | |
| 74 | 2 | 10 | — | 2.5 | — | — | 0 | 80 | 0 | — | 2 | 0 | 5 mM | — |
| | | | | | | | | | | | | | arginine | |
| 75 | 2 | 10 | — | 2.5 | — | — | 0 | 50 | 0 | — | 0 | 1 | 5 mM | — |
| | | | | | | | | | | | | | arginine | |
| 76 | 2 | 10 | — | 2.5 | — | — | 0 | 55 | 0 | — | 2 | 1 | 0 | — |
| 77 | 2 | 0 | — | 0 | — | — | 0 | 90 | 0 | — | 0 | 0 | 10 mM | — |
| | | | | | | | | | | | | | arginine | |
| 78 | 2 | 0 | — | 3.75 | — | — | 0 | 20 | 0 | — | 0 | 2 | 10 mM | — |
| | | | | | | | | | | | | | arginine | |
| 79 | 2 | 0 | — | 3.75 | — | — | 0 | 85 | 0 | — | 0 | 0 | 0 | — |
| 80 | 2 | 20 | — | 0 | — | — | 0 | 35 | 0 | — | 4 | 2 | 0 | — |
| 81 | 2 | 20 | — | 3.75 | — | — | 0 | 20 | 0 | — | 0 | 2 | 0 | — |
| 82 | 2 | 20 | — | 0 | — | — | 0 | 25 | 0 | — | 0 | 2 | 10 mM | — |
| | | | | | | | | | | | | | arginine | |
| 83 | 2 | 10 | — | 2.5 | — | — | 0 | 65 | 0 | — | 0 | 0.5 | 5 mM | — |
| | | | | | | | | | | | | | arginine | |
| 84 | 2 | 0 | — | 2.5 | — | — | 0 | 55 | 0 | — | 0 | 1 | 5 mM | — |
| | | | | | | | | | | | | | arginine | |
| 85 | 2 | 0 | — | 2.5 | — | — | 0 | 55 | 0 | — | 2 | 1 | 5 mM | — |
| | | | | | | | | | | | | | arginine | |
| 86 | 2 | 20 | — | 3.75 | — | — | 0 | 75 | 0 | — | 0 | 0 | 0 | — |
| 87 | 2 | 20 | — | 0 | — | — | 0 | 85 | 0 | — | 4 | 0 | 0 | — |
| 88 | 2 | 40 | — | 5 | — | — | 0 | 40 | 5 | — | 0 | 0 | 10 mM | — |
| | | | | | | | | | | | | | arginine | |
| 89 | 2 | 0 | — | 2.5 | — | — | 131 | 0 | 5 | — | 0 | 0 | 0 | — |
| 90 | 2 | 0 | — | 0 | — | — | 0 | 95 | 0 | — | 0 | 0 | 0 | — |
| 91 | 2 | 0 | — | 0 | — | — | 0 | 0 | 0 | — | 0 | 0 | 0 | — |
| 92 | 2 | 0 | — | 0 | | — | 0 | | 0 | — | 0 | 0 | 0 | — |

*Me-β-CD is Methyl β cyclodextrin (Wacker, Munich, Germany)
**DDPC is didecanoyl L-α-phosphoatidylcholine (NOF Corp., White Plains, NY)
***EDTA is edetate disodium (JTBaker, Phillipsburg, NJ)
****MP/PP is methyl paraben sodium/propyl paraben sodium (Spectrum, Gardena, CA)

pH was measured using a Cole Parmer semi-micro NMR tube glass pH probe with Orion 520Aplus pH meter (Thermo Electron Corp, Waltham, Mass.). The pH was adjusted using 2N HCL or 2N NaOH as necessary to meet the parameters specified in the formulation.

Osmolality was measured with an advanced multichannel osmometer, Model 2020 (Advanced Instruments, Inc., Norwood, Mass.).

Tracheal/bronchial epithelial cell membrane inserts (EpiAirway, MatTek Corp., Ashland, Mass.) were received the day before the experiment. Each tissue insert was placed in a well of a 6 well plate which contained 0.9 ml of serum free media and cultured at 37° C. for 24 hours to allow the tissues to equilibrate. The day of the experiment, transepithelial electrical resistance measurements were taken for each insert using a Tissue Resistance Measurement Chamber connected to an Epithelial Voltohmeter (World Precision Instruments, Inc., Sarasota, Fla.).

After the background transepithelial electrical resistance was determined, 1 ml of media was placed in the bottom of each well in a six well plate. The inserts were inverted and drained and placed into new wells with fresh media. For samples 1-12, 100 μl of the formulation to be tested was then added to an insert. For samples 13-92, 25 μl of the formulation was added to each insert. The inserts were placed in a shaking incubator at 100 rpm and 37° C. for one hour. The tissue inserts were then removed from the incubator. 200 μl of fresh media was placed in each well of a 24 well plate and the inserts were transferred. The basolateral solution remaining in the six well plate after removal of the insert was harvested and stored at 2-8° C. until it was assayed by EIA (Oxytocin Enzyme Immunoassay Kit: High Sensitivity, Peninsula Laboratories Inc, San Carlos, Calif.). Formulation 5 had a permeation of 21.2%. Formulation 1, 2, 3, and 4 had permeations of 15.7%, 19.7%, 9.6% and 17.9%, respectively. These permeation levels are a significant increase over the permeation of carbetocin without enhancer excipients. The permeation of carbetocin alone (in just buffer and salt) is less than 1.0%.

200 μl of fresh media was gently added to each tissue insert in the 24 well plate and the plate was placed no a shaker table at room temperature for 5 minutes. 150 μl of apical solution was removed from each insert and reserved for a lactase dehydrogenase assay. The inserts were then washed with 300 μl of media; 300 μl of new media was added to each insert, the inserts were incubated for 20 minutes at room temperature and the transepithelial electrical resistance was measured.

The inserts are then transferred into a new 24 well plate containing no media and the appropriate amount of media was added to the apical surface in order to total 300 μl. The inserts were then shaken for five minutes at 100 RPM at room temperature. 50-100 μl of the apical media were then removed, placed in 0.5 to 1.5 tubes and kept at 2-8° C. until needed.

The samples were then centrifuged at 1000 rpm for 5 minutes. 2 μl of the supernatant was removed and added to a 96 well plate. 48 μl of media was then used to dilute the supernatant to make a 25× dilution and each sample was assayed in triplicate for LDH loss using a CytoTox 96 Cytotoixcity Assay Kit (Promega Corp., Madison, Wis.).

For analysis of the basolateral media, 50 μl of the reserved 150 μl solution was loaded into a 96 well assay plate and assayed in triplicate.

Cell viability was assessed using an MTT assay kit (MatTek Corp., Ashland, Mass.). MTT concentrate was thawed and diluted with media at a ratio of 2 ml MTT:8 ml media. 300 μl MTT-media mix was added to each well of a 24 well plate. Tissue culture inserts were drained and transferred to the MTT containing well and incubated at 37° C. in the dark for three hours. After incubation, each insert was removed from the plate, and then immersed in the wells of a fresh 24-well plate containing 2 ml extractant solution. The plate was then covered and incubated overnight at room temperature in the dark. The liquid in each insert was then decanted back into the well from which it was taken and the insert was discarded. 50 μl of the extractant solution from each well was then pipetted in triplicate into a 96 well plate and diluted with the addition of 150 μl of fresh extractant solution. The optical density of the samples was then measured at 550 nm on a Spectramax plate reader (Molecular Devices, Sunnyvale, Calif.) using SpectraPro software.

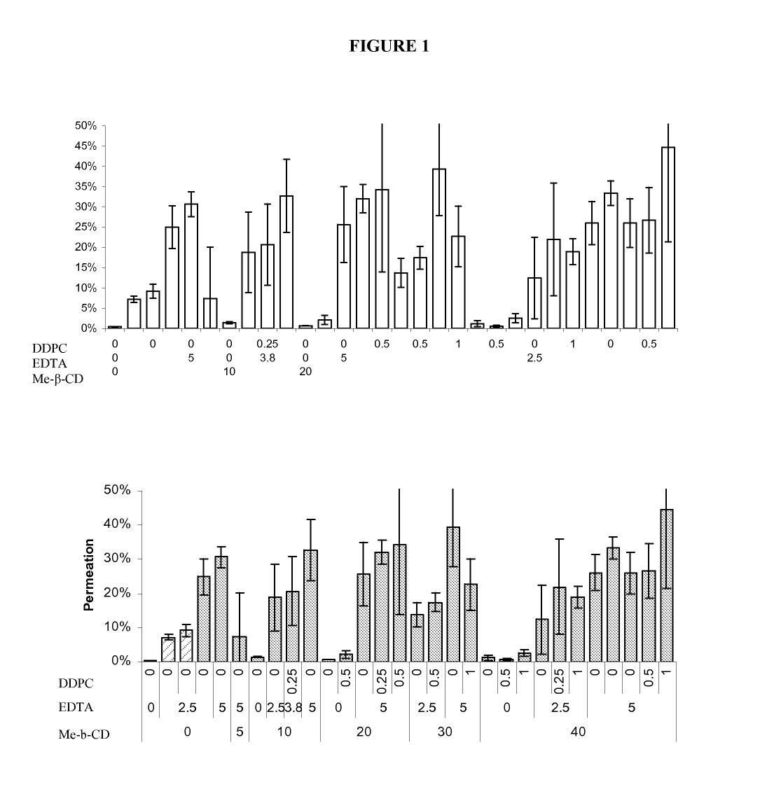

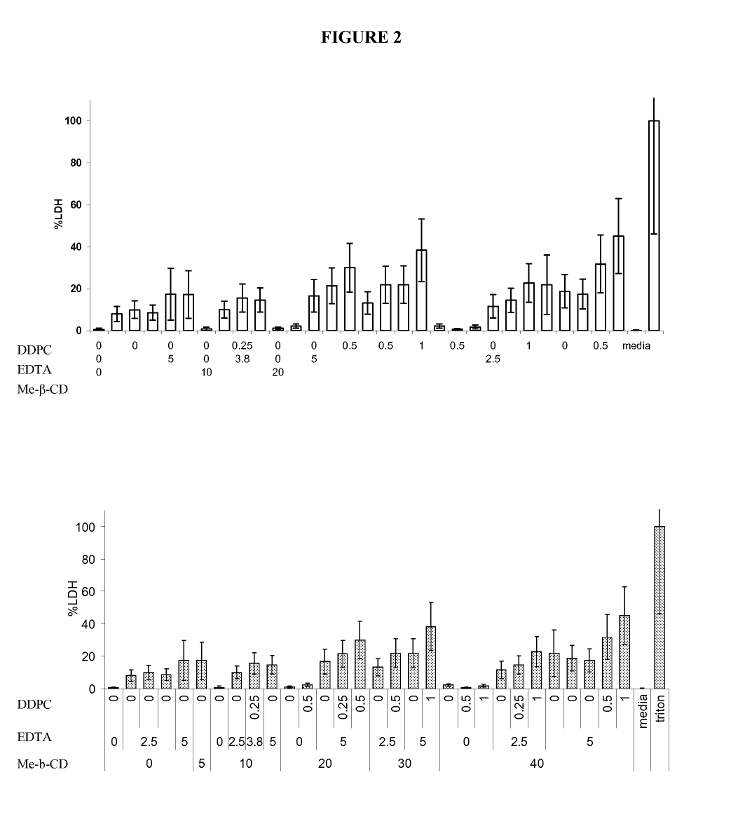

As can be seen in FIG. 1 of samples 13-44, EDTA was a significant factor in increasing permeation and sorbitol appears to reduce permeation of carbetocin. The optimal formulations as predicted by DOE included EDTA and Me-b-CD. Additionally, as shown in FIG. 2, EDTA was the most significant factor in cytotoxicity. In combination with Me-β-CD and EDTA, ethanol also enhanced permeation.

### Example II

### Pharmacokinetics in Rabbits

Rabbits were treated with carbetocin by intranasal administration of pharmaceutical compositions. The following formulations were tested:

| | | | | | | | | | Carbetocin |
|---|---|---|---|---|---|---|---|---|---|
| | Carbetocin | Me-β-CD | EDTA | Arginine | Sorbitol | NaCl | CB | | % Label |
| Group # | (mg/ml) | (mg/ml) | (mg/ml) | (mM) | (mM) | (mM) | (mg/ml) | pH | Claim |
| 1 | 0.03 | 0 | 0 | 10 | 0 | 150 | 0 | 7 | 87.1 |
| 2 | 2 | 0 | 3.5 | 10 | 0 | 57 | 5 | 4 | 101.2 |
| 3 | 2 | 10 | 3.5 | 10 | 0 | 52 | 5 | 4 | 110.2 |
| 4 | 2 | 10 | 3.5 | 10 | 104 | 0 | 5 | 4 | 103.0 |
| 5 | 2 | 20 | 3.5 | 10 | 0 | 50 | 5 | 4 | 102.0 |
| 6 | 4 | 10 | 3.5 | 10 | 0 | 52 | 5 | 4 | 99.2 |

The following results were obtained from measurements of mean blood levels:

PK Data:

| | | | Tmax | Cmax | AUClast |
|---|---|---|---|---|---|
| Group # | Formulation | Dose (μg/kg) | (min) | (pg/mL) | (min * pg/mL) |
| 1 | IM | 3 | 13 | 4522.80 | 171874.50 |
| 2 | IN | 30 | 29 | 1244.80 | 46724.50 |
| 3 | IN | 30 | 27 | 1098.80 | 67283.50 |
| 4 | IN | 30 | 30 | 692.80 | 32378.00 |
| 5 | IN | 30 | 27 | 1678.20 | 51911.50 |
| 6 | IN | 60 | 30 | 3090.40 | 169038.00 |

% F:

| Group # | Formulation | Dose (μg/kg) | AUClast (min * pg/mL) | % Bio |
|---|---|---|---|---|
| 1 | IM | 3 | 171874.50 | N/A |
| 2 | IN | 30 | 46724.50 | 2.72 |
| 3 | IN | 30 | 67283.50 | 3.91 |
| 4 | IN | 30 | 32378.00 | 1.88 |
| 5 | IN | 30 | 51911.50 | 3.02 |
| 6 | IN | 60 | 169038.00 | 4.92 |

% CV:

| | | | Tmax | Cmax | AUClast |
|---|---|---|---|---|---|
| Group # | Formulation | Dose (μg/kg) | (min) | (pg/mL) | (min * pg/mL) |
| 1 | IM | 3 | 21.07 | 13.37 | 16.73 |
| 2 | IN | 30 | 42.93 | 101.09 | 67.41 |
| 3 | IN | 30 | 24.85 | 30.94 | 42.83 |
| 4 | IN | 30 | 0.00 | 27.25 | 33.13 |
| 5 | IN | 30 | 24.85 | 46.69 | 51.75 |
| 6 | IN | 60 | 35.36 | 27.64 | 15.86 |

These results show a carbetocin bioavailabily of about 4-5% can be achieved by the pharmaceutical formulations of the invention.

### Example III

### Anxiolytic Effect of Carbetocin and Oxytocin as Determined by the Elevated Plus Maze Assay

Sixty male rats obtained from the Charles River laboratories are divided into six groups of ten animals each. All animals are maintained in compliance with the standards of the National Research Council and are fed certified rodent diet (Teklad, Madison, Wis.) and water ad libitum. The animals are acclimated to their housing for a minimum of 5 days prior to their first day of dosing.

Following acclimation, the animals will be administered vehicle, alprazolam, oxytocin or carbetocin respectively according to the following schedule.

TABLE 2
Group Assignments and Dose Levels

| | Number | | | | | |
|---|---|---|---|---|---|---|
| | of | | | | | Con- |
| | Animals | | | Dose | Volume | centration |
| Group | Males | Route | Treatment | (mg/kg) | (mL/kg) | (mg/mL) |
| 1 | 10 | ICV* | Vehicle | 0 | 0.03 | 0 |
| 2 | 10 | IP** | Alprazolam | 0.5 | 5 | 0.1 |
| 3 | 10 | ICV | Oxytocin | 0.05 | 0.03 | 1.7 |
| 4 | 10 | IM*** | Oxytocin | 1.0 | 0.2 | 5 |
| 5 | 10 | ICV | Carbetocin | 0.25 | 0.03 | 8.3 |
| 6 | 10 | IM | Carbetocin | 5 | 0.2 | 25 |

*intracerebroventricular administration
**intraperitoneal administration
***intramuscular administration

The rats are evaluated using an elevated plus maze (Holmes, A., et al., Behav. Neurosci. 115(5):1129-44, October 2001) prior to treatment to establish a baseline. Groups 1, 3, and 5 are tested 20 minutes after administration of the treatment and groups 2, 4, and 6 are tested thirty minutes after treatment using an elevated plus maze.

The elevated plus maze consists of a platform with 4 arms, two open and two closed (50×10×50 cm enclosed with an open roof). Rats are tested two at a time and placed by hand in the center of the platform of two separate mazes, at the crossroad of the 4 arms, facing one of the open arms. After fifteen minutes, the first rat is left for a few seconds until the second rat's fifteen minutes is completed. The rats are monitored remotely.

The rats are evaluated for time spent in open arm exploration, time spent in closed arm exploration and scored for anxiety according to the percent of time spent in open arm exploration ([time spent in open arms/time spent in open arms+time spent in closed arms]×100); the absolute time spent in open arm exploration, and the percent of open arm entries. ([number of open arm entries/number of open arm entries+number of closed arm entries]×100. The number of total arm entries are used as a measure of overall locomotor activity. The number and location of fecal boli will also be calculated. The scores are compared to the vehicle controls and to the baseline using one-way ANOVA followed by the appropriate post-hoc test (Bonferroni/Dunnets) and a p<0.05 is considered to be statistically significant.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

SAHUQUE, L.; E. F. KULLBERG; A. J. McGEEHAN; J. R. KINDER; M. P. HICKS; M. G. BLANTON; P. H. JANAK; and M. F. OLIVE, “Anxiogenic and aversive effects of corticotropin-releasing factor (CRF) in the bed nucleus of the stria terminalis in the rat: role of CRF receptor subtypes,” Psychopharmacology 186(1):122-132, Berl., 2006.
CARVALHO, M. C.; L. ALBRECHET-SOUZA; S. MASSON; and M. L. BRAND\check{A}O, “Changes in the biogenic amine content of the prefrontal cortex, amygdala, dorsal hippocampus, and nucleus accumbens of rats submitted to single and repeated sessions of the elevated plus-maze test,” Braz. J. Med. Biol. Res. 38(12):1857-66, 2005.
LANGEN, B.; U. EGERLAND; K. BERNÖSTER; R. DOST; K. UNVERFERTH; and C. RUNDFELDT, “Characterization in Rats of the Anxiolytic Potential of ELB139 [1-(4-Chlorophenyl)-4-piperidin-1-yl-1,5-dihydro-imidazol-2-on], a New Agonist at the Benzodiazepine Binding Site of the GABAA Receptor,” J. Pharmacol. Exp. Ther. 314:717-724, 2005.

What is claimed is:

1. A method of preventing or treating autism spectrum disorders in a mammalian subject comprising administering an effective amount of oxyctocin or an oxytocin analog to said subject.

2. The method of claim 1, wherein the oxytocin analog is 4-threonine-1-hydroxy-deaminooxytocin, 9-deamidooxytocin, an analog of oxytocin containing a glycine residue in place of the glycinamide residue, 7-D-proline-oxytocin (2,4-diisoleucine)-oxytocin, an analog of oxytocin with natriuretic and diuretic activities, deamino oxytocin analog; a long-acting oxytocin (OT) analog, 1-deamino-1-monocarba-E12-[Tyr(OMe)]-OT(dCOMOT), carbetocin, (1-butanoic acid-2-(O-methyl-L-tyrosine)-1-carbaoxytocin, deamino-1 monocarba-(2-O-methyltyrosine)-oxytocin [d(COMOT)]), [Thr4-Gly7]-oxytocin (TG-OT), oxypressin, Ile-conopressin, atosiban, deamino-6-carba-oxytoxin (dC60), d[Lys(8)(5/6C-Fluorescein)]VT, d[Thr(4), Lys(8)(5/6C-Fluorescein)]VT, [HO(1)][Lys(8)(5/6C-Fluorescein)]VT, [HO(1)][Thr(4), Lys(8)(5/6CFluorescein)]VT, d[Om(8)(5/6C-Fluorescein)]VT, d[Thr(4), Om(8)(5/6C-Fluorescein)]VT, [HO(1)][Om(8)(5/6C-Fluorescein)]VT, [HO(1)][Thr(4), Om(8)(5/6C-Fluorescein)]VT, desmopressin, and 1-deamino-oxytocin in which the disulfide bridge between residues 1 or 6 is replaced by a thioether.

3. The method of claim 1, wherein the oxytocin analog is carbetocin.

4. The method of claim 1, further comprising a secondary or adjunctive therapeutic agent.

5. The method of claim 4, wherein the therapeutic agent is administered to said subject in a coordinate administration protocol, simultaneously with, prior to, or after, administration of the oxytocin or oxytocin analog to said subject.

6. The method of claim 4, wherein the secondary or adjunctive therapeutic agent is selected from serotonin reuptake inhibitors, selective serotonin reuptake inhibitors antipsychotic medications, anti-convulsants, stimulant medications, anti-virals, axiolytic medications and immunotherapy.

7. The method of claim 4, wherein the additional or adjunctive therapeutic agent is a vitamin.

8. The method of claim 1 further comprising an adjunctive therapy.

9. The method of claim 8, wherein the adjunctive therapy is behavioral modification or diet modification.

10. The method of claim 1, wherein the oxytocin or oxytocin analog is formulated with a solubilizer and chelator.

11. The method of claim 10, wherein the solubilizer is methyl-β-cyclodextrin.

12. The method of claim 10, further comprising a salt as a tonicifier.

13. The method of claim 10, wherein the chelator is edetate disodium.

14. A method of treating or preventing one or more symptoms of autism spectrum disorders comprising administering an effective amount of oxyctocin or an oxytocin analog to said subject.

15. The method of claim 14, wherein the symptom is social withdrawal, eye contact avoidance, repetitive behaviors, anxiety, attention deficit, hyperactivity, depression, loss of speech, verbal communication difficulties, aversion to touch, visual difficulties, comprehension difficulties, and sound or light sensitivity.

16. The method of claim 14, wherein the oxytocin analog is 4-threonine-1-hydroxy-deaminooxytocin, 9-deamidooxytocin, an analog of oxytocin containing a glycine residue in place of the glycinamide residue, 7-D-proline-oxytocin (2,4-diisoleucine)-oxytocin, an analog of oxytocin with natriuretic and diuretic activities, deamino oxytocin analog; a long-acting oxytocin (OT) analog, 1-deamino-1-monocarba-E12-[Tyr(OMe)]-OT(dCOMOT), carbetocin, (1-butanoic acid-2-(O-methyl-L-tyrosine)-1-carbaoxytocin, deamino-1 monocarba-(2-O-methyltyrosine)-oxytocin [d(COMOT)]), [Thr4-Gly7]-oxytocin (TG-OT), oxypressin, Ile-conopressin, atosiban, deamino-6-carba-oxytoxin (dC60), d[Lys(8)(5/6C-Fluorescein)]VT, d[Thr(4), Lys(8)(5/6C-Fluorescein)]VT, [HO(1)][Lys(8)(5/6C-Fluorescein)]VT, [HO(1)][Thr(4), Lys(8)(5/6CFluorescein)]VT, d[Om(8)(5/6C-Fluorescein)]VT, d[Thr(4), Om(8)(5/6C-Fluorescein)]VT, [HO(1)][Om(8)(5/6C-Fluorescein)]VT, [HO(1)][Thr(4), Om(8)(5/6C-Fluorescein)]VT, desmopressin, and 1-deamino-oxytocin in which the disulfide bridge between residues 1 or 6 is replaced by a thioether.

17. The method of claim 14, wherein the oxytocin analog is carbetocin.

18. The method of claim 14, further comprising a secondary or adjunctive therapeutic agent.

19. The method of claim 18, wherein the therapeutic agent is administered to said subject in a coordinate administration protocol, simultaneously with, prior to, or after, administration of the oxytocin or oxytocin analog to said subject.

20. The method of claim 18, wherein the secondary or adjunctive therapeutic agent is selected from serotonin reuptake inhibitors, selective serotonin reuptake inhibitors antipsychotic medications, anti-convulsants, stimulant medications, anti-virals, axiolytic medications and immunotherapy.

21. The method of claim 18, wherein the additional or adjunctive therapeutic agent is a vitamin.

22. The method of claim 14 further comprising an adjunctive therapy.

23. The method of claim 22, wherein the adjunctive therapy is behavioral modification or diet modification.

24. The method of claim 14, wherein the oxytocin or oxytocin analog is formulated with a solubilizer and chelator.

25. The method of claim 24, wherein the solubilizer is methyl-β-cyclodextrin.

26. The method of claim 24, further comprising a salt as a tonicifier.

27. The method of claim 24, wherein the chelator is edetate disodium.

28. A method of treating disorders related to autism spectrum disorders in a mammalian subject comprising administering an effective amount of oxytocin or an oxytocin analog to said subject.

29. The method of claim 28, wherein the related disorder is Landau-Kleffner Syndrome, Multi-systems disorder, social phobia, generalized anxiety disorder, panic disorder, posttraumatic stress disorder, phobia, agoraphobia, obsessive-compulsive disorder, paranoid personality disorder, schizotypal personality disorder, schizoid personality disorder, avoidant personality disorder, conduct disorder, borderline personality disorder, histrionic personality disorder; repetitive disorders, impulse control and addiction disorders, eating disorders, dementia, Alzheimer's, Creutzfeld-Jakob disease, attention deficit disorder, attention deficit hyperactivity disorder, mild cognitive decline, or cognitive disorder not otherwise specified.

30. The method of claim 28, wherein the oxytocin analog is 4-threonine-1-hydroxy-deaminooxytocin, 9-deamidooxytocin, an analog of oxytocin containing a glycine residue in place of the glycinamide residue, 7-D-proline-oxytocin (2,4-diisoleucine)-oxytocin, an analog of oxytocin with natriuretic and diuretic activities, deamino oxytocin analog; a long-acting oxytocin (OT) analog, 1-deamino-1-monocarba-E12-[Tyr(OMe)]-OT(dCOMOT), carbetocin, (1-butanoic acid-2-(O-methyl-L-tyrosine)-1-carbaoxytocin, deamino-1 monocarba-(2-O-methyltyrosine)-oxytocin [d(COMOT)]), [Thr4-Gly7]-oxytocin (TG-OT), oxypressin, Ile-conopressin, atosiban, deamino-6-carba-oxytoxin (dC60), d[Lys(8)(5/6C-Fluorescein)]VT, d[Thr(4), Lys(8)(5/6C-Fluorescein)]VT, [HO(1)][Lys(8)(5/6C-Fluorescein)]VT, [HO(1)][Thr(4), Lys(8)(5/6CFluorescein)]VT, d[Om(8)(5/6C-Fluorescein)]VT, d[Thr(4), Om(8)(5/6C-Fluorescein)]VT, [HO(1)][Om(8)(5/6C-Fluorescein)]VT, [HO(1)][Thr(4), Om(8)(5/6C-Fluorescein)]VT, desmopressin, and 1-deamino-oxytocin in which the disulfide bridge between residues 1 or 6 is replaced by a thioether.

31. The method of claim 28, wherein the oxytocin analog is carbetocin.

32. The method of claim 28, further comprising a secondary or adjunctive therapeutic agent.

33. The method of claim 32, wherein the therapeutic agent is administered to said subject in a coordinate administration protocol, simultaneously with, prior to, or after, administration of the oxytocin or oxytocin analog to said subject.

34. The method of claim 32, wherein the secondary or adjunctive therapeutic agent is selected from serotonin reuptake inhibitors, selective serotonin reuptake inhibitors antipsychotic medications, anti-convulsants, stimulant medications, anti-virals, axiolytic medications and immunotherapy.

35. The method of claim 32, wherein the additional or adjunctive therapeutic agent is a vitamin.

36. The method of claim 28 further comprising an adjunctive therapy.

37. The method of claim 36, wherein the adjunctive therapy is behavioral modification or diet modification.

38. The method of claim 28, wherein the oxytocin or oxytocin analog is formulated with a solubilizer and chelator.

39. The method of claim 38, wherein the solubilizer is methyl-α-cyclodextrin.

40. The method of claim 38, further comprising a salt as a tonicifier.

41. The method of claim 38, wherein the chelator is edetate disodium.

42. A composition for preventing or treating autism spectrum disorders comprising autism spectrum disorders in a mammalian subject comprising an effective amount of oxytocin or an oxytocin analog.

43. The composition of claim 42, wherein the oxytocin analog is 4-threonine-1-hydroxy-deaminooxytocin, 9-deamidooxytocin, an analog of oxytocin containing a glycine residue in place of the glycinamide residue, 7-D-proline-oxytocin (2,4-diisoleucine)-oxytocin, an analog of oxytocin with natriuretic and diuretic activities, deamino oxytocin analog; a long-acting oxytocin (OT) analog, 1-deamino-1-monocarba-E12-[Tyr(OMe)]-OT(dCOMOT), carbetocin, (1-butanoic acid-2-(O-methyl-L-tyrosine)-1-carbaoxytocin, deamino-1 monocarba-(2-O-methyltyrosine)-oxytocin [d(COMOT)]), [Thr4-Gly7]-oxytocin (TG-OT), oxypressin, Ile-conopressin, atosiban, deamino-6-carba-oxytoxin (dC60), d[Lys(8)(5/6C-Fluorescein)]VT, d[Thr(4), Lys(8)(5/6C-Fluorescein)]VT, [HO(1)][Lys(8)(5/6C-Fluorescein)]VT, [HO(1)][Thr(4), Lys(8)(5/6CFluorescein)]VT, d[Om(8)(5/6C-Fluorescein)]VT, d[Thr(4), Om(8)(5/6C-Fluorescein)]VT, [HO(1)][Om(8)(5/6C-Fluorescein)]VT, [HO(1)][Thr(4), Om(8)(5/6C-Fluorescein)]VT, desmopressin, and 1-deamino-oxytocin in which the disulfide bridge between residues 1 or 6 is replaced by a thioether.

44. The composition of claim 42, wherein the oxytocin analog is carbetocin.

45. The composition of claim 42, wherein the composition further comprises a solubilizer, and chelator.

46. The composition of claim 45, wherein the solubilizer is methyl-β-cyclodextrin.

47. The composition of claim 45, further comprising a salt as a tonicifier.

48. The composition of claim 45, wherein the chelator is edetate disodium.

49. A composition for treating or preventing one or more symptoms of autism spectrum disorders comprising an effective amount of oxytocin or an oxytocin analog.

50. The composition of claim 49, wherein the symptom is social withdrawal, eye contact avoidance, repetitive behaviors, anxiety, attention deficit, hyperactivity, depression, loss of speech, verbal communication difficulties, aversion to touch, visual difficulties, comprehension difficulties, and sound or light sensitivity.

51. The composition of claim 49, wherein the oxytocin analog is 4-threonine-1-hydroxy-deaminooxytocin, 9-deamidooxytocin, an analog of oxytocin containing a glycine residue in place of the glycinamide residue, 7-D-proline-oxytocin (2,4-diisoleucine)-oxytocin, an analog of oxytocin with natriuretic and diuretic activities, deamino oxytocin analog; a long-acting oxytocin (OT) analog, 1-deamino-1-monocarba-E12-[Tyr(OMe)]-OT(dCOMOT), carbetocin, (1-butanoic acid-2-(O-methyl-L-tyrosine)-1-carbaoxytocin, deamino-1 monocarba-(2-O-methyltyrosine)-oxytocin [d(COMOT)]), [Thr4-Gly7]-oxytocin (TG-OT), oxypressin, Ile-conopressin, atosiban, deamino-6-carba-oxytoxin (dC60), d[Lys(8)(5/6C-Fluorescein)]VT, d[Thr(4), Lys(8)(5/6C-Fluorescein)]VT, [HO(1)][Lys(8)(5/6C-Fluorescein)]VT, [HO(1)][Thr(4), Lys(8)(5/6CFluorescein)]VT, d[Om(8)(5/6C-Fluorescein)]VT, d[Thr(4), Om(8)(5/6C-Fluorescein)]VT, [HO(1)][Om(8)(5/6C-Fluorescein)]VT, [HO(1)][Thr(4), Om(8)(5/6C-Fluorescein)]VT, desmopressin, and 1-deamino-oxytocin in which the disulfide bridge between residues 1 or 6 is replaced by a thioether.

52. The composition of claim 49, wherein the oxytocin analog is carbetocin.

53. The composition of claim 49, wherein the composition further comprises a solubilizer and chelator.

54. The composition of claim 53, wherein the solubilizer is methyl-β-cyclodextrin.

55. The composition of claim 53, further comprising a salt as a tonicifier.

56. The composition of claim 53, wherein the chelator is edetate disodium.
